(19) United States
(12) Patent Application Publication
(10) Pub. No.: US 2026/0348833 A1
(43) Pub. Date: Nov. 5, 2026

(54) ASSEMBLY AND METHOD FOR DETERMINING THE CONCENTRATION OF ONE OR MORE SUBSTANCES IN A LIQUID OR GASEOUS MEDIUM

(71) Applicant: Fraunhofer-Gesellschaft zur Förderung der angewandten Forschung e.V., München (DE)

(72) Inventors: Thorsten GRAUNKE, Erlangen (DE); Sebastian HETTENKOFER, Erlangen (DE); Stephan JUNGER, Erlangen (DE); Jens-Uwe GARBAS, Erlangen (DE); Ruslan RYBALKO, Erlangen (DE); Wladimir TSCHEKALINSKIJ, Nürnber (DE)

(73) Assignee: Fraunhofer-Gesellschaft zur Förderung der angewandten Forschung e.V., München (DE)

(21) Appl. No.: 19/124,554

(22) PCT Filed: Oct. 19, 2023
(86) PCT No.: PCT/EP2023/079101

§ 371 Date: Apr. 25, 2025

(30) Foreign Application Priority Data
Oct. 27, 2022 (DE) 10 2022 128 472.7

(51) Int. Cl. G01N 21/17; G01N 21/03; G01N 21/05; G01N 21/3504; G01N 21/3577; G01N 21/85; G01N 33/14

(52) U.S. Cl. CPC G01N 21/1702; G01N 21/0332; G01N 21/05; G01N 21/3504; G01N 21/3577; G01N 21/85; G01N 33/146; G01N 2021/052; G01N 2021/1704; G01N 2021/8571; G01N 2201/06146; G01N 2201/0668

(57) ABSTRACT

An assembly for determining the concentration of one or more substances in a liquid or gaseous medium includes a measuring chamber into which the medium can be introduced or through which the medium can flow, as well as a number of measuring channels, each formed by a light source and one or more detectors associated with only this light source. The number of the measuring channels corresponds with at least the number of a selection of characteristic absorption peaks that are at least required for determining the concentration of the one or more substances. Each measuring channel has at least one acoustic detector to measure an absorption of optical radiation in the liquid or gaseous medium in the measuring chamber for another characteristic absorption peak of the selection, as the light source of this measuring channel emits narrow-band radiation corresponding to a wavelength of the respective absorption peak.

### TECHNICAL APPLICATION FIELD

The present invention relates to an assembly for determining the concentration of one or more substances in a liquid or gaseous medium which have characteristic absorption peaks when illuminated with optical radiation. The invention also relates to a method for determining the concentration with an assembly of such kind.

Analysis of liquid or gaseous media with regard to the concentration of individual substances in such media is an important aspect of many technical and scientific applications. For example, many substances have characteristic optical absorption peaks, which constitute a fingerprint of the respective substance, thus enabling the substances to be identified or determined uniquely by means of optical absorption spectroscopy. Photoacoustic spectroscopy can also be used to determine the concentration of substances in a liquid or gaseous medium. In the case of photoacoustic spectroscopy, the specific light absorption at an absorption peak causes a pressure change or acoustic wave in the medium, which is captured with a detector and converted into an electrical signal. The detector may have the form of a microphone, for example. The captured pressure change is a measure of the concentration of the corresponding substance. However, for each measurement the wavelength of the irradiated optical radiation must be adapted to substance or substances that is/are to be measured. For this purpose, the required wavelength can be adjusted with the aid of a spectral filter, for example, or by using a tunable laser.

### PRIOR ART

Thus for example, EP 3508836 B1 describes a photoacoustic gas sensor in which radiation from a broadband IR-light source is directed through a bandpass filter and into a measuring chamber containing the gas that is to be measured. The optical bandpass filter only allows a certain part of the light spectrum to pass through. The central wavelength is adapted to the absorption maximum of the gas that is to be detected. Time modulation of the IR light source up to 100 Hz gives creates a sound wave inside the measuring chamber through the absorption of the radiation in the gas, which sound wave is measured with a highly sensitive pressure sensor on the measuring chamber and serves as a measure of the concentration of the absorbing gas. However, the gas sensor in such an assembly is only capable of detecting a gas with an absorption maximum at the corresponding filter wavelength.

DE 102021108745 A1 describes an assembly for multispectral light emission and a multispectral sensor equipped therewith, with which it is possible to make adjustments and changes to the emitted wavelengths easily and quickly. This assembly has a broadband light source in combination with a filter array consisting of Several spectral filters and a switching device for controlling the passage of the light emitted by the light source through individual filters of this filter array. This makes it possible to adapt or vary the wavelength or spectral distribution of the emitted optical radiation according to the number and characteristics of the various filters in the filter array. In this way, the spectral distribution of the emitted light can also be adapted to the respective application, including to characteristic absorption peaks in the context of optical absorption spectroscopy, for example, or photoacoustic spectroscopy for determining the concentration of substances in a medium. In this context, the assembly includes one or more broadband light source, a filter array, a switching device and a common detector.

Particularly for the selective analysis of complex samples in the sub-ppb range, many substances must be determined as to their concentration in a liquid or gaseous medium with a high degree of sensitivity. To the extent possible, this process should be as inexpensive as possible. One example of such is an analysis of the wine flavour, in which a large number of flavouring agents in the wine are to be determined. The problem to be addressed by the present invention consists in specifying an assembly and a method for determining the concentration of one or more substances in a liquid or gaseous medium, which can be implemented inexpensively and can be adapted to the respective application.

### PRESENTATION OF THE INVENTION

The problem is solved with the assembly and the method according to claims 1 and 12. Advantageous variants of the assembly and of the method are the subject matter of the dependent claims or may be derived from the following description and exemplary embodiments.

The suggested assembly is designed for the purpose of determining the concentration of one or more substances, in particular pure substances, in a liquid or gaseous medium, which have characteristic absorption peaks when illuminated with optical radiation. The assembly comprises at least one measuring chamber, into which the liquid or gaseous medium can be introduced or through which the liquid or gaseous medium can flow, as well as a number of measuring channels, which are each formed by a light source and one or more detectors associated with only this light source. The number of measuring channels corresponds to at least the number of a selection of the characteristic absorption peaks that are at least required for the determining the concentration of the one or more substances in the liquid or gaseous medium. This makes use of the fact that in order to carry out a unique identification and determination of the concentration of a substance, in many cases it not necessary to measure all characteristic absorption peaks of said substance, on the contrary it is sufficient to measure a smaller number of absorption peaks, in other words a selection of the characteristic absorption peaks. Each measuring channel has at least one acoustic detector as the detector, or one of the detectors, and is designed such that it measures an absorption of optical radiation in the liquid or gaseous medium in the measuring chamber for one of the characteristic absorption peaks of the selection. For this purpose, the light source of this measuring channel is selected such that it emits narrow-band radiation at a wavelength corresponding to the wavelength of the respective absorption peak. In this context, the term narrow-band is understood to mean that the bandwidth of the emitted radiation is small enough to enable only one of the characteristic absorption peaks of the substances in the liquid or gaseous medium to be captured in each case. The bandwidth of the emitted radiation of the respective light source preferably has a value of <300 nm, particularly preferably <100 nm. Each measuring channel is designed to measure another characteristic absorption peak of the selection concerned.

For the purposes of the present patent specification, the light source is understood to be a light emitting unit that emits narrow-band optical radiation at the corresponding wavelength. This may be a single light emitter, for example a laser diode or a light emitting diode (LED), which emits the narrow-band radiation directly. But the light source may also be formed by a light emitter that emits wide-band light with one or more upstream spectral filters, wherein the one or more filters then has/have the corresponding narrow-band passband at the desired wavelength. Additional optical elements, such as lenses or optical fibres, may also be part of the light source.

The suggested design of the assembly means that it can easily be adapted to the respective measurement task, i.e. to configure it specifically for the substances that are to be determined in the liquid or gaseous medium by selecting the required number and emission wavelength of each of the measuring channels. This modular principle and the independence of the individual measuring channels enable an inexpensive and simple structure. The assembly can also be used to inexpensively measure more complex samples, in wine flavour analysis for example.

In the suggested assembly, each measuring channel is designed for a specific wavelength. At the same time, only one acoustic detector for photoacoustic spectroscopy or also multiple detectors with different measurement principles can be used for each measuring channel. Particularly preferably, a combination of photoacoustic spectroscopy (PAS) and absorption spectroscopy is used in the suggested assembly. In principle, besides the acoustic detectors, in particular pressure detectors or microphones, various types of detectors can be used in the measuring channels, for example microbolometers, pyroelectrical detectors, resistive detectors (MOX), thermal conductivity detectors (TCD), heat tone detectors or photodetectors. Depending on the light source and detector, such detectors may also be integrated in the light source or light emitter. This is true particularly for detector types in which a heating element is required (e.g., MOX, WLD, pellistor).

Depending on the detector, these are arranged inside the measuring chamber, as in the case of a microphone or pressure sensor, but they may also be arranged outside the measuring chamber, as in the case of a photodetector, for example. Of course, a photodetector may also be arranged inside the measuring chamber. The measuring chamber itself must be designed so that it is permeable to optical radiation at least at the corresponding coupling in or coupling out points for such radiation.

In the preferred variant, the measuring chamber is of tubular design and is connected to a pump, via which the liquid or gaseous medium to be measured is drawn or pumped. The corresponding measuring channels are then arranged next to each other along the length of the tubular measuring chamber. In one particular variant, the tubular measuring chamber, which may also have a non-circular, for example rectangular cross-section, may have multiple tubular extensions along its longitudinal extension, each extending transversely to the longitudinal axis of the measuring chamber, and into which the medium to be measured diffuses as it flows through the measuring chamber. Extensions of such kind are also possible in the case of a measuring chamber that is not of tubular construction, each forming separate measurement volumes. The measuring channels are then arranged on each of these extensions, as will be described in greater detail in a subsequent embodiment. A membrane or a filter may also be present at the transition to these extensions, which only allows certain substances to diffuse into the extensions. In this way, it is possible to assure a prior separation of the substances.

In an advantageous variant, at least one of the light emitters of the measuring channels is formed by a heating element, i.e. a radiant heater that emits radiation in the IR range. In a particularly preferable variant, this radiant heater serves at the same time as a thermal conductivity sensor by measuring the change in its electrical resistance in the respective measuring channel. In such case, the measuring channel has a diffusion orifice at the corresponding location in the region of this light emitter, through which a small fraction of the medium flowing through the measuring chamber is diffused out and directed over the light emitter. The diffusion orifice may also have the form of an optical filter, if such filter is designed accordingly, as a plasmonic filter, for example. In this way, it is possible to detect CO₂ or hydrogen for example with this light emitter. Alternatively, an additional sensor may also be arranged close to the light emitter designed as a radiant heater, for example to form a MOX sensor, a thermal conductivity sensor or a heat tone sensor, which is then heated via the light emitter, meaning that it does not need its own heating element. In this case, the medium then flows over this additional sensor, again with the result that for example CO₂ or hydrogen can be detected.

The variant described above, in which a radiant heater serves as a light emitter and at the same time is used as a thermal conductivity sensor or in conjunction with an additional MOX sensor, thermal conductivity sensor or heat tone sensor, may also be utilised independently of the present assembly as a measurement device, which for example comprises only one light source, or also an assembly consisting of multiple light sources and/or measuring channels that share a common detector.

With the suggested method, the suggested assembly is designed such that the individual measuring channels are adapted to the selection of absorption peaks of the substances that are to be measured with regard to number and light sources. The liquid or gaseous medium is then introduced into the measuring chamber or sucked or pumped through the measuring chamber. In each case, the absorption at the corresponding wavelength or the respective characteristic absorption peak is measured with the detectors of the measuring channels in order to derive the concentrations of the corresponding substances therefrom. Preferably, a temporally serial measurement is performed, in which the individual measuring channels are operated at different times, to prevent the individual measuring channels from interfering with each other, so that only one measuring channels is active at any one time, that is to say only the light source of this one measuring channel is switched on in each case. In another variant, the light sources of the individual measuring channels are modulated with a different frequency to generate acoustic waves at differing frequencies, which do not interfere with each other. This also prevents the individual measuring channels from interfering with each other if they are operated simultaneously.

The suggested assembly and the associated method can be used in many application fields, for example in medicine, in the environmental sector, in process engineering and civil security. This includes for example the analysis of industrial processes and parameters (process monitoring), quality assurance, early fire detection, flavour analysis, detection of off-odours, breathing gas analysis, safety applications, environmental analyses, an application as electronic nose or electronic tongue. Of course, this is not an exhaustive list.

### BRIEF DESCRIPTION OF THE DRAWING

In the following text, the suggested assembly and the associated method will be explained again in greater detail with reference to exemplary embodiments in conjunction with the drawing. In the drawing:

FIG. 1 shows an example of characteristic absorption peaks of EtOH and ethyl acetate;

FIG. 2 shows an example of characteristic absorption peaks of CO and CO₂;

FIG. 3 shows a first example of a variant of the suggested assembly;

FIG. 4 shows an example of a particular variant of a measuring channel;

FIG. 5 shows a further example of a particular variant of a measuring channel;

FIG. 6 shows a further example of a variant of the suggested assembly;

FIG. 7 shows a further example of a variant of the suggested assembly;

FIG. 8 shows a schematic representation of a time-offset operating mode of the individual measuring channels in a variant of the suggested assembly;

FIG. 9 shows a schematic representation of an activation of different measuring channels with different frequencies;

FIG. 10 shows two further examples of the variant of a measuring channel;

FIG. 11 shows a further exemplary variant of the suggested assembly; and

FIG. 12 shows an exemplary arrangement of the light sources and the additional sensor in the variant of FIG. 5.

### WAYS OF IMPLEMENTING THE INVENTION

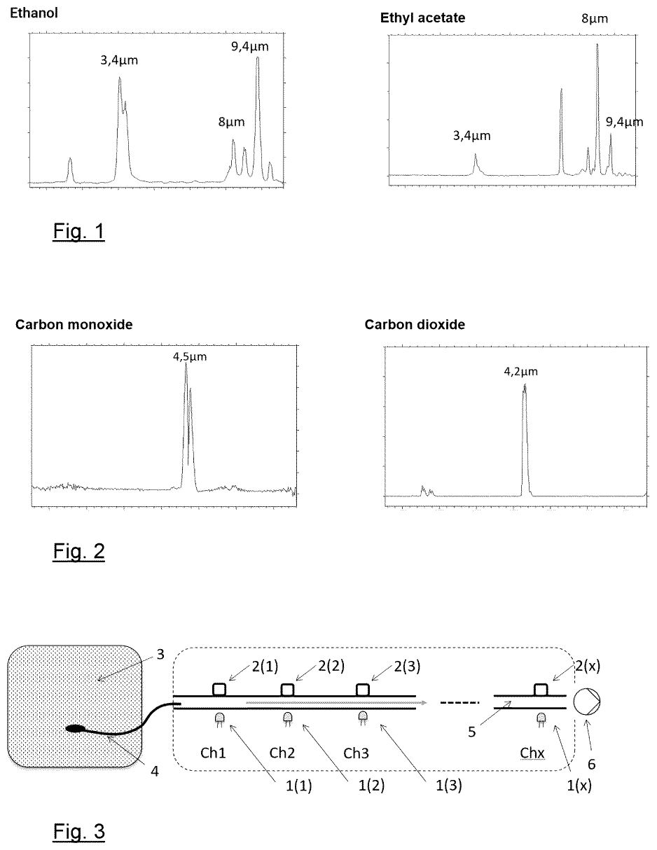

With the suggested assembly for determining individual substances in a liquid or gaseous medium, use is made of the fact that the individual substances have one or more characteristic optical absorption peaks (the “fingerprint”), by means of which they can be identified uniquely, and via which their concentration in a medium can also be determined. For this purpose, FIG. 1 shows an example of characteristic absorption peaks of EtOH (ethanol) and ethyl acetate. The panel on the left shows the characteristic absorption peaks of ethanol, the panel on the right represents the characteristic absorption peaks of ethyl acetate. In order to differentiate these two substances, it is sufficient to carry out a measurement at three different wavelengths, at 3.4 μm, 8 μm and 9.4 μm. Accordingly, with the suggested assembly the concentrations of these two substances in a liquid or gaseous medium can be determined with three measuring channels having a narrow-band light source with a central wavelength of 3.4 μm, 8 μm and 9.4 μm respectively. The concentrations of the two substances can then be determined by using the absorption strength captured with the detector of the respective measuring channel and the ratio of this absorption strength at the three wavelengths. In this context, the suggested assembly and associated method function on the basis that the individual characteristic absorption peaks of the substances to be determined are known.

FIG. 2 shows a further example of characteristic absorption peaks of CO (carbon monoxide) and CO₂ (carbon dioxide). As may be seen in the panel on the left, CO has a characteristic absorption peak at a wavelength of 4.5 μm auf. The panel on the right shows that CO₂ has a characteristic absorption peak at a wavelength of 4.2 μm. Therefore, the measurement at two wavelengths (4.5 μm and 4.2 μm) is sufficient to differentiate and determine the concentrations of these two substances. For this purpose, the suggested assembly accordingly has only two measuring channels for these two wavelengths.

FIG. 3 shows an example of a possible variant of the suggested assembly. Here, the measuring chamber 5 is of tubular construction. The individual measuring channels (Ch1, Chx) are each arranged side by side along the measuring chamber. Each measuring channel (Ch1, . . . Chx) in this example consists of a light source (1(1), 1(x)) and a detection unit (2(1) . . . 2(x)). Each light source (1(1), . . . 1(x)) is tuned to a certain wavelength, which corresponds to the wavelength of a characteristic absorption peak of the substance that is to be determined. The individual light sources (1(1), . . . 1(x)) may each consist of a narrow-band light source (e.g., laser diode, LED) or a broadband light source with one or more upstream optical filter(s). The detection unit (2(1) . . . 2(x)) may comprise one or more detectors. A PAS detector is used as the detector or one of the detectors. If there are multiple detectors per measuring channel, an optical absorption detector may also be used as well. The individual measuring channels (Ch1, . . . Chx) may be arranged side by side in modular manner. The media passed through the measuring chamber 5 are not converted in the respective measuring channel.

Depending on the measuring principle, the detectors may be arranged inside and/or outside the measuring chamber 5. During the measurement, the sample is sucked out of a sample reservoir 3 through the measuring chamber with a sampling system 4 by means of a pump 6. The measurement is carried out with the individual measuring channels while the sample is flowing through the measuring chamber 5.

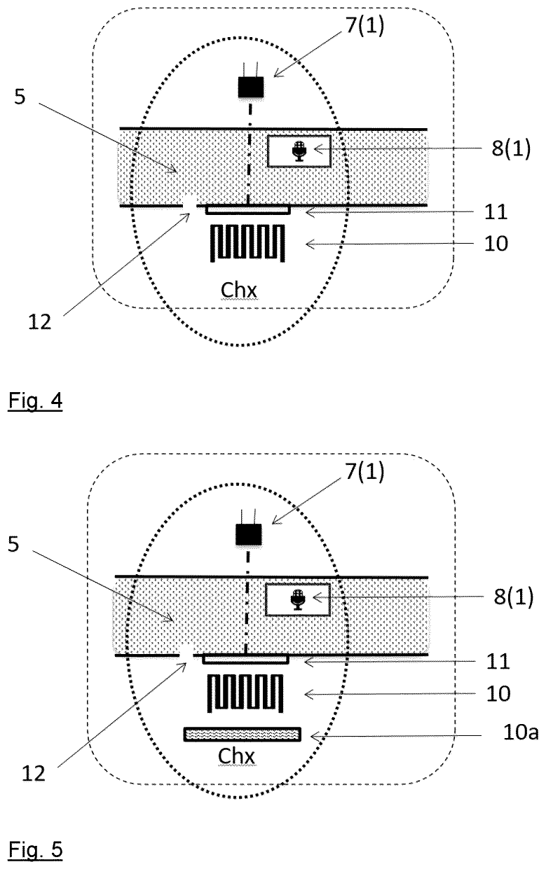

The light sources (1(1), . . . 1(x)) of the measuring channels (Ch1, . . . Chx) may be of various designs. FIG. 4 shows an example of a specially designed measuring channel, which again is tuned to a certain wavelength. In this example, a heating element 10 is used as broadband IR-light emitter. This heating element 10 is combined with an optical filter 11 that only allows optical radiation at the corresponding wavelength to pass through. In this example, the heating element 10 also functions as detector. For this purpose, the medium flowing through the chamber 5 is permitted to diffuse via corresponding diffusion orifices 12 on the measuring chamber 5 and over the heating element 10. A resistance measurement at the heating element 10 enables said element to function as a thermal conductivity detector, for detecting CO₂ or hydrogen for example. The heating element 10 thus functions as both an IR emitter and a thermal conductivity detector. An optical absorption detector 7(1) is positioned opposite the heating element 10 on the measuring chamber 5. Additionally, a microphone 8(1) is arranged inside the measuring chamber 5, close to the location where an absorption takes place, so that a PAS measurement can be performed as well as the optical absorption measurement.

FIG. 5 shows an alternative variant to FIG. 4, in which it is not the heating element 10 that functions as additional detector, but rather another sensor 10a beside or below the heating element 10. This additional Sensor 10a may be for example a MOX sensor, a thermal conductivity sensor or a heat tone sensor (each without its own heating element), which is heated by the heating element 10 in the form of an IR emitter. In this example, the medium passing out through the diffusion orifice 12 thus flows over this additional sensor 10a, so that for example CO₂ or hydrogen can be detected.

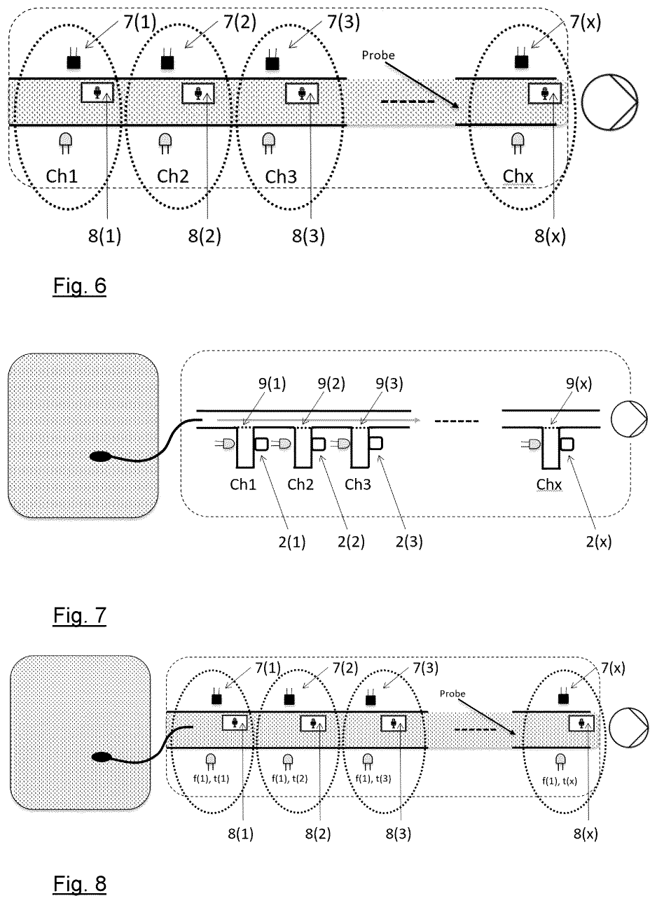

FIG. 6 shows a further exemplary variant of the suggested assembly, in which the measuring chamber is again of tubular construction, and the individual measuring channels (Ch1, Chx) are arranged along said measuring chamber. Here too, the medium is sucked in through the measuring chamber by means of a pump 6. In this example, the individual measuring channels (Ch1, . . . Chx) each have a corresponding light source, an oppositely positioned optical absorption detector (7(1), . . . 7(x)), and a microphone (8(1), . . . 8(x)) as PAS detector, and are arranged along the horizontally positioned measuring chamber.

The measuring chamber itself may also include tubular extensions designed specifically for the measurement, as is illustrated in the example of FIG. 7. These extensions along the tubular measuring chamber form separate measurement volumes, on which the respective measuring channels are arranged. Here too, the medium is preferably sucked out of a reservoir with a pump. The concentration equalisation in the individual extensions is assured by diffusion through a membrane and/or a filter (9(1) . . . 9(x)). Optionally, a gas-selective membrane or a gas-selective filter, made for example from thermoplastic polycondensate such as PEEK, PEI, or a fluoropolymer such as FEP, ECTFE or PFA, may also be integrated in the respective extension to increase the selectivity of the measurement. A membrane or filter of such kind may also be used at the diffusion orifices 12 in the variants shown in FIGS. 4 and 5. The membrane or filter may then be used for prior separation in the gas phase-in the case of a gaseous medium—, or to measure gas that is dissolved in the liquid—in the case of a liquid medium. Again, the individual measuring channels each include a light source and one or more detectors, as was shown earlier in the previous exemplary embodiments.

If several measuring channels are used as in the suggested assembly with PAS detector, and the light is modulated in the individual measuring channels with the same frequency f(1), there is a risk of mutual disturbance between the individual channels, since the acoustic waves generated may possibly interfere with each other. In one version of the suggested method, therefore, the individual measuring channels are operated one after the other, so that only one measuring channel is ever active at any point in time (t(1), . . . t(x)), which is to say only one light source is switched on at any given time. This is represented schematically in FIG. 8. In such a case, the individual measuring channels may be operated in any order.

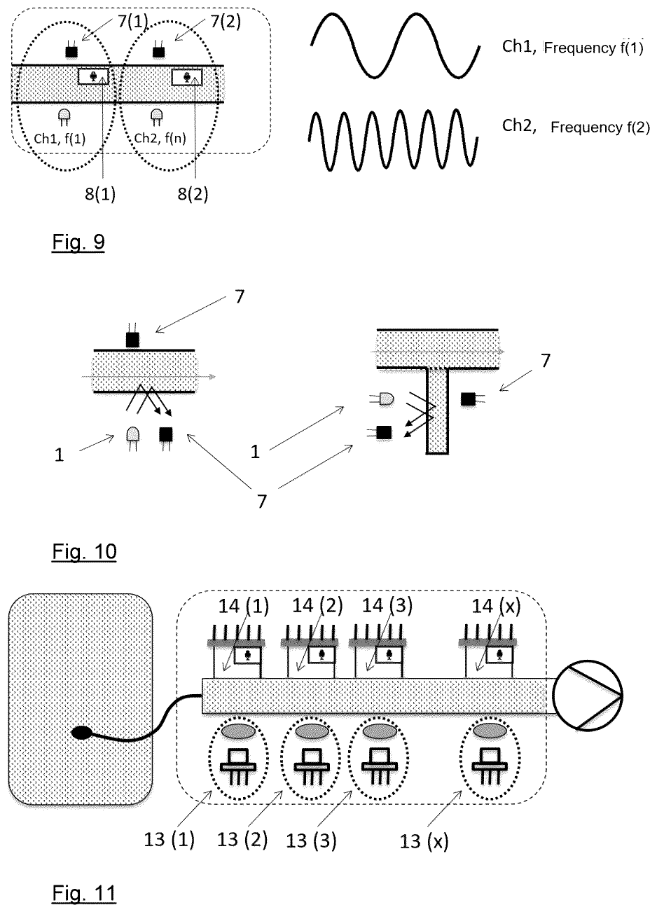

Alternatively, however, the light sources of the individual measuring channels may also be modulated at different frequencies (f(1), . . . f(n)). This enables the simultaneous operation of all measuring channels, with the result that all measuring channels are active, since acoustic waves created with differing frequencies do not interfere with each other. This is represented schematically in FIG. 9 for two measuring channels Ch1, Ch2, which shows a schematic illustration of the modulation signals for the light sources of the two channels Ch1, Ch2 in the panel on the right. The acoustic waves induced thereby do not interfere with each other, as they are modulated differently. With additional electronics, the interfering signal of the respective other channel can be filtered out at each of the detectors 8(19, 8(2). This operating mode may also be combined with the operating mode of FIG. 8.

When photodetectors are used additionally, that is to say the optical absorption of the light emitted by the light source is measured directly, the measurement can be performed on both the reflected and transmitted light. This is represented in both panels of FIG. 10 for the different variants described previously. In this example, the respective measuring channel also includes two photodetectors 7 for each light source 1. Only the absorption detector on the opposite side needs direct illumination and must be located correspondingly opposite the light source 1. The second detector, for measuring reflected light, may be arranged beside the light source 1, on the same side of the measuring chamber or measurement volume, as is shown schematically in both panels.

Finally, FIG. 11 again shows an exemplary variant of the suggested assembly, in which the PAS detector (14(1) . . . 14(x)) (pressure sensor, for example microphone) is arranged outside the measuring chamber but is coupled to it acoustically. The narrow-band light source (13(1) . . . 13(x)) in this example also includes an optical system, for example a collimating lens, as is suggested schematically in the figure. In this example too, the medium to be measured is again sucked out of a reservoir via a pump.

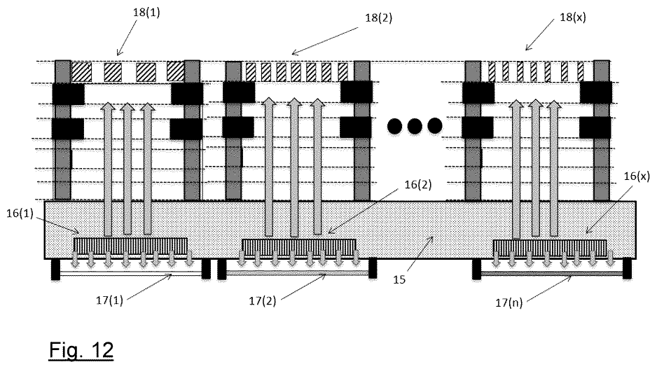

FIG. 12 shows an exemplary arrangement of multiple light sources with additional sensors of the variant of FIG. 5, in particular the miniaturisation of the optical filter 11, the heating element 10 and the additional sensor 10a from FIG. 5. The miniaturisation may be done in semiconductor technology. All other elements of the measuring channel shown in FIG. 5 remain unchanged.

In the arrangement of FIG. 12, there is an array of microheaters (16(1) . . . 16(x)) on the one side of the wafer 15. A MOX sensor, a thermal conductivity sensor or a heat tone sensor for example may be integrated underneath the respective heater 16 as additional sensor 17. These sensors 17(1) . . . 17(x) may be of the same type. But differing sensor principles or heaters without an additional sensor may also be combined. On the other side of the wafer 15 there is an optical filter array with multiple filters (18(1) . . . 18(x); e.g., plasmonic filters). The heater 16 emits broadband radiation through the wafer 15 towards the optical filters 18. The functional principle of the front side of this arrangement has already been described in DE 10 2021 108 745 A1 in conjunction with FIG. 4 of this patent disclosure. The additional sensors 17 on the rear side are new.

### LIST OF REFERENCE NUMERALS

1 Light source
2 Detection unit
3 Reservoir
4 Sampling system
5 Measuring chamber
6 Pump
7 Optical absorption detector
8 PAS-detector
9 Membrane, Filter
10 IR-heating element
10a Additional sensor
11 Filter
12 Diffusion orifice
13 Light source with optical system
14 PAS detector
15 Wafer
16 Micro-heating element
17 Additional sensor
18 Filter
Ch Measuring channel

1. Assembly for determining the concentration of one or more substances in a liquid or gaseous medium, which have characteristic absorption peaks when illuminated with optical radiation, consisting of at least
a measuring chamber, into which the liquid or gaseous medium can be introduced or through which the liquid or gaseous medium can flow, and
a number of measuring channels, each of which is formed by a light source and one or more detectors associated only with this light source,
wherein the number of the measuring channels corresponds with at least the number of a selection of characteristic absorption peaks that are at least required for determining the concentration of the one or more substances in the liquid or gaseous medium,
wherein the measuring channels each have at least one acoustic detector as detector(s), and
wherein each measuring channel is designed such that it measures an absorption of optical radiation in the liquid or gaseous medium in the measuring chamber for one of the characteristic absorption peaks of the selection by as the light source of this measuring channel emits narrow-band radiation that corresponds to a wavelength of the respective absorption peak.

2. Assembly according to claim 1,
characterized in that
the measuring channels also have at least one optical detector as detector(s).

3. Assembly according to claim 1,
characterized in that
the measuring chamber is connected to a pump, by which the liquid or gaseous medium can be transported through the measuring chamber.

4. Assembly according to claim 1,
characterized in that
the measuring chamber is of tubular construction.

5. Assembly according to claim 4,
characterized in that
the measuring channels are arranged side by side along the measuring chamber.

6. Assembly according to claim 1,
characterized in that
the light sources are formed by narrow-band light emitters or by broadband light emitters with upstream narrow-band optical filter(s).

7. Assembly according to claim 6,
characterized in that
at least one of the broadband light emitters is a heating element that is arranged outside the measuring chamber and emits radiation in the IR range.

8. Assembly according to claim 7,
characterized in that
the measuring chamber has at least one diffusion orifice in the region of the heating element, through which the liquid or gaseous medium can diffuse over the heating element, wherein at the same time the heating element is designed as a thermal conductivity detector.

9. Assembly according to claim 7,
characterized in that
an additional sensor is arranged on the heating element, in particular to create a MOX sensor, a thermal conductivity sensor or a heat tone sensor, and the measuring chamber has at least one diffusion orifice in the region of the heating element, through which the liquid or gaseous medium can diffuse over the additional sensor.

10. Assembly according to claim 1,
characterized in that
the measuring chamber has a plurality of tubular extensions that form separate measurement volumes, wherein a measuring channel is arranged on each of these extensions.

11. Assembly according to claim 10,
characterized in that
the extensions are separated from the main volume of the measuring chamber by a membrane or filter which only allows certain substances to diffuse into the extensions.

12. Method for determining the concentration of one or more substances in a liquid or gaseous medium, which have characteristic absorption peaks upon illumination with optical radiation, with an assembly according to claim 1, in which
the liquid or gaseous medium is introduced into the measuring chamber or is sucked or pumped through the measuring chamber,
on which a number of the measuring channels is arranged that at least corresponds to the number of a selection of the characteristic absorption peaks, which is at least required for determining the concentration of the one or more substances in the liquid or gaseous medium,
wherein each measuring channel is designed such that its light source emits narrow-band radiation at a wavelength that corresponds to a wavelength of one of the absorption peaks of the selection, and
the absorption at the characteristic absorption peaks of the selection is measured with the measuring channels

13. Method according to claim 12,
characterized in that
the measurement is performed with the measurement principle of photoacoustic spectroscopy.

14. Method according to claim 13,
characterized in that
the measurement principle of optical absorption spectroscopy is also used for the measurement.

15. Method according to claim 12,
characterized in that
the measurement is performed with the individual measuring channels in temporal sequence.

16. Method according to claim 12
characterized in that
the radiation emitted by the light sources of the measuring channels is modulated with different frequencies when the measurement principle of photoacoustic spectroscopy is used.

17. Method according to claim 12 for determining the concentration of flavour components of a product, in particular of wine.
